(19) United States
(12) Patent Application Publication
(10) Pub. No.: US 2012/0058993 A1
(43) Pub. Date: Mar. 8, 2012

(54) Stable Suspension Formulation

(75) Inventors: Peter William Surman, Henderson (NZ); Sharon Ferguson, Hobsonville (NZ); Wai Bik Mak, Mt. Eden (NZ); Andrew Douglas McLeod, West Harbour (NZ); Praneeta Sharma, Henderson (NZ); Sunitha de Costa, Penrose (NZ)

(73) Assignee: DOUGLAS PHARMACEUTICALS LTD., Auckland (NZ)

(21) Appl. No.: 13/269,794

(22) Filed: Oct. 10, 2011

Related U.S. Application Data
(63) Continuation-in-part of application No. 10/561,930, filed on May 23, 2006, now Pat. No. 8,057,811.

(30) Foreign Application Priority Data
Jul. 23, 2003 (NZ) 527142

(51) Int. Cl. A61K 31/5513; A61P 25/18

(52) U.S. Cl. 514/220

(57) ABSTRACT

A physicochemically stable aqueous composition including clozapine suspension.

This application is a continuation in part of U.S. patent application Ser. No. 10/561,930, filed May 23, 2006, which is a National Stage Entry of PCT/NZ04/00158, filed Jul. 22, 2004, which claims the benefit of NZ 527142, filed Jul. 23, 2003.

### TECHNICAL FIELD

The present invention is directed to a stable suspension formulation of clozapine for oral administration and to processes for preparing such formulations.

### BACKGROUND TO THE INVENTION

Clozapine (8-chloro-11-(4-methyl-1-piperazinyl-5H-dibenzo[b,e][1,4] diazepine) is a well-known compound having anti-psychotic activity. Details about this compound are disclosed in monograph 2448 of the 13^{th} edition of the Merck Index, the disclosure of which is hereby incorporated by way of reference.

Currently there are no liquid formulations of clozapine commercially available and, as a result, hospital pharmacists are often required to compound liquid formulations using crushed clozapine tablets for patients who have difficulty in swallowing or who feign ingestion.

Clozapine is insoluble in water and therefore the logical option for preparing a liquid formulation is to form it into an aqueous suspension. However, when clozapine is simply added directly to water, the compound settles rapidly to form a dense cake at the base of the aqueous mixture. The caking cannot easily be redistributed and as such would potentially compromise the accuracy of drug dose delivered to a patient.

In order to overcome this, a standard formulation technique would be to use a suitable wetting agent, to promote flocculation. Flocculation is a process where suspended particles agglomerate, forming larger particles that settle loosely and can be readily re-dispersed with gentle shaking thus overcoming the caking problem.

Clozapine is generally regarded as a stable molecule. But, surprisingly, when clozapine is formed into an aqueous suspension with a wetting agent and other formulating agents as might be considered standard in the art, the suspended active was found to be readily susceptible to hydrolysis which was indicated by a marked pH change on extended storage. As a result, the accuracy of the drug dose delivered to the patient could again be compromised.

There would be a clear advantage to be able to provide a physicochemically stable suspension formulation of clozapine for oral administration which would retain its physico-chemical stability over a reasonable storage period. Such a product characteristic would be important to the production of a commercial liquid formulation.

### SUMMARY OF THE INVENTION

In broad terms the invention in a first aspect may be seen to comprise a physicochemically stable aqueous composition including clozapine in suspension.

In broad terms, the invention may be seen to comprise a physicochemically stable aqueous composition including clozapine in suspension together with a wetting agent, wherein the pH of the composition is maintained in the range of about 6 to about 11. In a further aspect, the invention comprises a physicochemically stable aqueous composition including clozapine in suspension together with a wetting agent, wherein the pH of the composition is within the range of about 5.6 to about 11. In a further aspect, the invention comprises a physicochemically stable aqueous composition including clozapine in suspension together with a wetting agent, wherein the pH of the composition is maintained in the range of about 5.6 to about 11.

In a further aspect the invention may be seen to comprise a physicochemically stable aqueous composition comprising clozapine in suspension and an agent capable of controlling and maintaining the pH of the composition, wherein the pH of the composition is maintained in the range of about 5.6 to about 11.

Preferably, the pH is maintained within the desired range using a buffer system.

Preferably the buffer system is a sodium phosphate/sodium hydroxide buffer system.

Preferably the pH is maintained in the range of from about 6 to about 8. In a preferred embodiment, the pH is maintained in the range from about 6 to about 7. In a preferred embodiment, the pH is maintained at about 6.5. In a preferred embodiment, the pH is maintained at about 6.7. In a preferred embodiment, the pH is maintained at about 6.9. In a further aspect, the invention comprises a method for preparing a physicochemically stable aqueous formulation including clozapine in suspension including the step of controlling the pH of the formulation between about 5.6 and about 11. In a preferred embodiment, the pH is maintained in the range from about 5.6 to about 8. In a preferred embodiment, the pH is maintained in the range from about 5.9 to about 7.

Preferably the amount of clozapine in the composition is from about 0.1% to about 10% by weight based on the total volume of the composition. In a preferred embodiment, the amount of clozapine in the composition is about 5% w/v. In a further embodiment the amount of clozapine is about 5% by weight based on the total weight of the composition. In a preferred embodiment the amount of clozapine is about 4.5% w/w. In a preferred embodiment the amount of clozapine in the composition is about 2.5% w/w.

Preferably the wetting agent is present in the composition in an amount of between about 0.1% and about 15% w/v. Preferably the wetting agent is present in an amount of between about 0.1% and about 19% w/v.

Preferably the wetting agent is selected from a suitable polyalcohol, such as propylene glycol, glycerin, or polyethylene glycol.

Preferably the composition includes polyvinyl pyrrolidone (PVP) as a crystal growth inhibitor.

In a preferred embodiment the PVP is present in an amount of between about 0.1% and 2.0% by weight based on the total volume of the composition. In a preferred embodiment the PVP is present in an amount of between about 0.005% to 0.1% w/v.

In a preferred embodiment the PVP is present in an amount of between about 0.01% and 1.0% w/v. In a preferred embodiment the PVP is present in an amount of between about 0.01% and 2.0% w/v. In a preferred embodiment the PVP is present in an amount of about 0.01% w/v. In a preferred embodiment the PVP is present in an amount of about 1% w/v.

Preferably the composition includes a suspending agent and/or a preservative.

In a preferred embodiment the suspending agent is present in an amount of between about 0.1% and about 0.3% w/v. In a preferred embodiment the suspending agent is present in an amount of between about 0.2% to about 0.6% w/v. In a preferred embodiment the suspending agent is present in an amount of between about 0.1% and about 0.4% w/v. In a preferred embodiment the suspending agent is present in an amount of about 0.2% w/v. In a preferred embodiment the suspending agent is present in an amount of about 0.55% w/v. Preferably the suspending agent is present in an amount of between about 0.4% and about 2.0% w/v. Preferably the suspending agent is present in an amount of between about 0.2% and about 2.0% w/v.

Preferably the preservative is present in an amount of between about 0.1% and about 0.5% w/v.

Preferably the suspending agent is xanthan gum. In a preferred embodiment the xanthan gum is present in an amount of between about 0.1% to about 0.6% w/v. In a preferred embodiment the xanthan gum is present in an amount of between about 0.1% to about 0.4% w/v. In a preferred embodiment the xanthan gum is present in an amount of about 0.55% w/v. In a preferred embodiment the xanthan gum is present in an amount of about 0.35% w/v. In a preferred embodiment the xanthan gum is present in an amount of about 0.2% w/v.

Preferably the preservative is a mixture of methyl, propyl and butyl parabens, most preferably methyl and propyl parabens.

Preferably the composition further includes a sweetening agent and/or a flavouring substance.

Preferably the composition includes: clozapine, glycerine, sodium dihydrogen phosphate dihydrate/NaOH buffer, xanthan gum, methyl paraben, propyl paraben, butyl paraben, and water.

Preferably the composition includes PVP.

Preferably the composition includes about:

(a) 50 mg/mL clozapine;

(b) 40 mg/mL propylene glycol;

(c) 7.8 mg/mL sodium dihydrogen phosphate dihydrate, q.s. sodium hydroxide;

(d) 6.0 mg/mL xanthan gum;

(e) 2.0 mg/mL methyl paraben;

(f) 0.5 mg/mL butyl paraben;

(g) 0.5 mg/mL chlorhexidine gluconate;

(h) q.s. water to final volume.

Preferably the composition includes about:

(a) 50 mg/mL clozapine;

(b) 108 mg/mL glycerine;

(c) 3.9 mg/mL sodium dihydrogen phosphate dihydrate, q.s. sodium hydroxide;

(d) 5.2 mg/mL xanthan gum;

(e) 2.2 mg/mL methyl paraben;

(f) 0.2 mg/mL propyl paraben;

(g) 0.2 mg/mL butyl paraben;

(h) 10.8 mg/mL PVP;

(i) 86.4 mg/mL sucrose;

(j) q.s. water to final volume.

Preferably the composition comprises about:

(a) 50 mg/ml Clozapine;

(b) 150 mg/ml Sorbitol 70% crystallizing solution;

(c) 0.1 mg/ml Povidone K90;

(d) 7.8 mg/ml Sodium dihydrogen phosphate dihydrate;

(e) 0.48 to 0.66 mg/ml Sodium hydroxide;

(f) 2 mg/ml Sodium methylparaben;

(g) 0.2 mg/ml Sodium propylparaben;

(h) 182 mg/ml Glycerol;

(i) 2 mg/ml Xanthan gum;

(j) 605.24 mg/ml Water.

Preferably the composition comprises about:

(a) 50 mg/ml Clozapine;

(b) 150 mg/ml Sorbitol 70% crystallising solution;

(c) 10 mg/ml Povidone K90;

(d) 3.9 mg/ml Sodium dihydrogen phosphate dihydrate;

(e) 2 mg/ml Sodium methylparaben;

(f) 0.2 mg/ml Sodium propylparaben;

(g) 130 mg/ml Glycerol;

(h) 5.5 mg/ml Xanthan gum;

(i) 640.4 to 648.4 mg/ml Water;

(j) 0 to 4 mg/ml Sodium Hydroxide Solution (1M);

(k) 0 to 4 mg/ml Hydrochloric acid Solution (1M).

In a further aspect, the invention may be seen to comprise a method for preparing a physicochemically stable aqueous formulation including clozapine in suspension including the step of controlling the pH of the formulation between about 6 and about 11. In a preferred embodiment, the pH is maintained in the range from about 6 to about 7. In a preferred embodiment, the pH is maintained at about 6.3. In a preferred embodiment, the pH is maintained at about 6.9.

In a further aspect, the invention comprises a method for preparing a physicochemically stable aqueous formulation including clozapine in suspension including the step of controlling the pH of the formulation between about 5.6 and about 11. In a preferred embodiment the pH is maintained in the range from about 5.6 to about 8. In a preferred embodiment, the pH is maintained in the range from about 5.9 to about 7. In a preferred embodiment, the pH is maintained in the range from about 5.9 to about 7 with a buffer.

In a further aspect, the invention may be seen to comprise a method for producing a physicochemically stable aqueous composition including clozapine in suspension including the following steps:

(a) stirring the active ingredient clozapine with about three quarters of the propylene glycol ascribed to the batch;

(b) addition of the buffer salt (and optionally sweetening agents) dissolved in about half the volume of water ascribed to the batch with constant stirring;

(c) adjusting the pH value with the base component of the buffer with mixing;

(d) addition of the preservatives dissolved in the remaining propylene glycol;

(e) slow addition of the suspending agent with continuous stirring until the mixture thickens;

(f) further diluting the suspension with water to the desired end-volume.

In a further aspect, the invention may be seen to comprise a method for producing a physicochemically stable aqueous composition including clozapine in suspension including the following steps:

(a) stirring the active ingredient clozapine with about three quarters of the glycerine ascribed to the batch;

(b) addition of the buffer salt (and optionally sweetening agents) dissolved in about half the volume of water ascribed to the batch with constant stirring;

(c) adjusting the pH value with the base component of the buffer with mixing;

(d) addition of the preservatives dissolved in a small volume of water;

(e) slow addition of the suspending agent wetted with the remaining glycerine with continuous stirring until the mixture thickens;

(f) further diluting the suspension with water to the desired end-volume.

In a further aspect, the invention may be seen to comprise a method for producing a physicochemically stable aqueous composition including clozapine in suspension including the following steps:

(a) wetting Clozapine with glycerine;

(b) adding PVP solution (0.025% w/v) and additional water;

(c) adding 0.25 M buffer at pH 6.3 to the mixture;

(d) adding Semi hydrated xanthan gum/glycerol solution;

(e) adding Sorbitol and additional water;

(f) adding PVP solution (0.18% w/v) and additional water;

(g) adding Paraben/glycerine solution and additional water;

(h) wherein the pH is between about 6.7 and 7.

In a further aspect, the invention may be seen to comprise a method for producing a physicochemically stable aqueous composition including clozapine in suspension including the following steps:

(a) wetting Clozapine with glycerine;

(b) making a PVP solution at 0.025% w/v and adding to the mixture;

(c) adding additional water so that the concentration of PVP becomes 0.0125% to promote flocculation;

(d) adding 0.25 M buffer at pH 6.3 to the mixture to enhance flocculation;

(e) adding Sorbitol 70% crystallizing solution and xanthan gum;

(f) adding additional PVP as a 0.18% w/v solution;

(g) adding preservatives;

(h) The final concentration of PVP is 0.01% w/v and the buffer is 50 mM;

(i) wherein the pH of the final suspension is about 6.9.

Preferably the method includes the step of PVP addition.

Preferably the PVP is added as an aqueous solution following addition of the suspending agent.

### FIGURE

FIG. 1 shows the mean plasma concentration (ng/ml) versus time (hrs) of clozapine following the oral administration of 0.25 mg/ml suspension according to a preferred form of the invention and half a 25 mg Clozapine tablet at time zero.

### DETAILED DESCRIPTION

The present invention is broadly concerned with the preparation of a physicochemically stable aqueous composition including clozapine in suspension formulation of clozapine for oral administration.

Clozapine is generally regarded as a stable molecule which is practically insoluble in water. On addition of clozapine directly to water, the drug settles to form a dense cake at the base of the mixture which cannot be readily redistributed. When a wetting agent was used, the caking problem was overcome but it was surprisingly found that the active was readily susceptible to hydrolysis. As a result, the clozapine suspension was not suitable for commercial use as it could not be stored for a reasonable period to allow later, accurate, use.

Unexpectedly, it has been found that it is possible to impart considerable stability to an aqueous suspension of clozapine if the pH of the aqueous suspension is controlled and maintained at a level between about 6 and about 11, or at a level between about 5.6 and about 11. Preferably the pH will be maintained within the range of 6 to 9 and more preferably between 7 and 8. In a preferred embodiment, the pH is maintained in the range from about 6 to about 7. In a preferred embodiment, the pH is maintained in the range from about 5.9 to about 7. In a preferred embodiment, the pH is maintained at about 6.5. In a preferred embodiment, the pH is maintained at about 6.6. In a preferred embodiment, the pH is maintained at about 6.7. In a preferred embodiment, the pH is maintained at about 6.9. If the pH is not controlled and maintained within this range, the active degrades quite quickly. Once this is recognised the creation of a composition having a physicochemical stability suitable for developing a commercially viable aqueous suspension can be achieved.

In order to control the pH, a suitable buffer system should be used. Buffer systems comprise mixtures of appropriate amounts of conjugate bases of various organic acids adjusted to the desired pH value with NaOH or HCl. Examples of suitable bases include but are not limited to: sodium citrate, potassium citrate, sodium bicarbonate, potassium bicarbonate, sodium dihydrogen phosphate and potassium dihydrogen phosphate. The buffer should have sufficient capacity to remain in the desired pH range throughout the product shelf life. Such issues would be well known to the skilled person.

The preferred buffer system is sodium dihydrogen phosphate/sodium hydroxide.

It has been found that the initial concentration and pH of the buffer prior to addition to the composition can have a significant effect on the degree of flocculation which occurs in the final composition. When the preferred initial concentration and pH are used then on combination with clozapine flocculation is controlled and small floccules are formed. The end result is a stable suspension which retains pH in the desired range over the desired shelf life.

The preferred range in pH of the buffer before addition is between about 6.2 and 6.35 which results in a pH of the suspension on preparation of about 6.8 to 6.9. The concentration of the buffer prior to combining with clozapine is between about 0.1M and 0.5M. The most preferred concentration is about 0.24M or about 0.25M, ±5%.

The amount (w/v) of clozapine in the composition will be a suitable amount as will be known to the skilled person in the art. Ranges between 0.1% to 10%, preferably from 2.5% to 7.5% in particular 5% w/v (50 mg/mL) would be used. As will be known to the skilled person, simple dilution of the suspension could be used to deliver a required dosage amount to a patient as needed.

The composition will also preferably include a wetting agent selected from any one or more of propylene glycol, glycerin or polyethylene glycol and like compounds as would be known to the skilled person. The % range of wetting agent in the composition will preferably be between about 0.1% and 20%, more preferably between 1% and 15% w/v. The % range of wetting agent in the composition will preferably be between about 1% and 20% w/v. The % range of wetting agent in the composition will preferably be between about 5% and 19% w/v. In a preferred embodiment the amount of wetting agent in the composition will be about 18.2% w/v. In a preferred embodiment the amount of wetting agent in the composition will be about 13% w/v.

The oral suspension according to the present invention will preferably also include a preservative to prevent the growth of micro-organisms such as bacteria, yeasts and fungi. The preservative should also be physicochemically stable in the pH range of 6 to 11, preferably 6 to 9. Suitable preservatives could be selected from any one or more of: chlorhexidine; methyl paraben; propyl paraben; butyl paraben and their salts; diazolidinyl urea (GERMALL II®); quaternary compounds, e.g. benzalkonium chloride and cetylpyridinium chloride, phenyl ethyl alcohol and the like. The preservative system may also play a role in contributing to the stability of our aqueous formulation by reducing the formation of acidic metabolites. The preservatives may therefore in certain embodiments function as an agent or agents to control and/or maintain the pH of the composition within desired ranges. The concentration of preservatives may range from about 0.01% to about 0.5% w/v. In a preferred embodiment the concentration of the preservative is about 0.2% w/v. In a preferred embodiment the concentration of the preservative is about 0.02% w/v.

When preparing a formulation with the active suspended in an aqueous carrier it is often necessary to add a suspending/stabilizing agent or agents to prevent settling of the active material. Over time, the settling (even if ordinarily capable of redistribution) could lead to caking of the active to the inside walls of the product pack, leading to difficulties with redispersion and accurate dispensing. Suitable stabilising agents are the polysaccharide stabilisers such as xanthan, guar and tragacanth gums, as well as the cellulose derivatives HPMC (hydroxypropyl methylcellulose) and AVICEL® RC-591 (microcrystalline cellulose/sodium carboxymethyl cellulose). While CARBOPOL® (carboxyvinyl polymer) is also a stabilising agent of use in the formulating art, research has shown that when this is used in the present formulation a physicochemically stable composition is not achieved. In a preferred embodiment the suspending agent will be present in the composition at about 0.1% to about 2.0% w/w.

Polyvinylpyrrolidone (PVP) can also be referred to as a stabilising agent but, in this context, has been found to be more correctly referred to as a crystal growth inhibitor due to its particular effect in this regard.

In preferred compositions both PVP and another stabilising or suspending agent (eg xanthan gum) will be present.

The composition will preferably include polyvinylpyrrolidone (PVP) as a crystal growth inhibitor. In preferred embodiments, the PVP used is a relatively long chain PVP such as a PVP having a K value greater than 30. In preferred embodiments PVP K90 is used. Preferably the PVP will be present in an amount of between about 0.005% and 3.0% w/v. Preferably the PVP will be present in an amount of between 0.5% and 2.0% w/v. Inclusion of the PVP imparts a surprising level of additional physicochemical stability to the composition and is thus a most preferred option for preparing a commercially viable composition. In a preferred embodiment the PVP is present in an amount of between about 0.005% to 0.1% w/v. In a preferred embodiment the PVP is present in an amount of between about 0.01% and 2.0% w/v. In a preferred embodiment the PVP is present in an amount of about 0.01% w/v. In a preferred embodiment the PVP is present in an amount of about 1% w/v.

A variety of sweeteners and flavourings could also be added as desired and as known to the skilled person. Additives such as sucrose and/or banana flavouring, for example, could be added. Sucrose could be replaced by xylitol or sorbitol for example when the composition is for use with diabetics. In a preferred embodiment, the sweetening agent is present in an amount of between about 10% and 20% w/v. In a preferred embodiment the sweetening agent is present in an amount of about 15% w/v. In a preferred embodiment the sweetening agent is sorbitol. In a preferred embodiment the sweetening agent is sorbitol 70% crystallising solution.

In certain embodiments, the clozapine active may be suspended in a high concentration syrup of sweetening agent or sugar. Suitable sweeteners may include sucrose and/or sorbitol. A high sugar concentration syrup, for example more than about 45%, or at least about 60%, or at least about 66% sugar w/w, may reduce or eliminate the formation of acidic metabolites. The sweetener can therefore act as an agent to control and/or maintain pH within a desirable range. In certain embodiments , sugars may act as the only such agent, or may be used in conjunction with preservatives.

Compositions according to the present invention have been characterised by their improved physicochemical stability. The term “physicochemically stable”, or similar terms, refer to an aqueous suspension formulation wherein, after storage for a period of up to about three months at a temperature of 40° C., the residual amount of clozapine is 95% or more of the initial clozapine concentration.

The term clozapine as used herein, refers to the free base form and pharmaceutically acceptable acid addition salts thereof. Possible salts include, but are not limited to, inorganic salts such as phosphates, carbonates and organic salts such as citrate and acetate. The term addition salt also includes the solvates of clozapine including, but not limited to, hydrates and alcoholates.

The aqueous suspensions according to the present invention are well suited to dilution with acidic non-alcoholic beverages such as citrus drinks, soft-drinks and the like. This option aids the palatability of the liquid and may result in improved patient compliance. As stated earlier, the dilution requirements to achieve an effective clozapine dosage would be well within the knowledge of the skilled person in this particular art.

Preferred forms of the composition will include:

(a) clozapine;

(b) a solvent (eg water);

(c) a wetting agent to disperse the clozapine;

(d) a stabilizing agent;

(e) a buffer; and preferably

(f) PVP.

A particular oral composition according to the present invention will include:

(a) clozapine;

(b) a suitable wetting agent to disperse the drug substance;

(c) a suitable buffer to control the pH in the range of 6 to 9;

(d) a stabilizing agent;

(e) a preservative;

(f) water; and preferably;

(g) PVP.

Preferably the clozapine would be present in an amount of between about 0.1% and 10%; the stabilising agent between about 0.5% and 2%; and the preservative between about 0.1% and 0.5% (all w/v).

A more preferred oral composition according to the present invention includes (all percentages w/v):

(a) 5.0% clozapine;

(b) 4.0% propylene glycol;

(c) 0.78% sodium dihydrogen phosphate dihydrate and sufficient sodium hydroxide to adjust the pH range from 6 to 9;

(d) 8.0% sucrose;

(e) 0.60% xanthan gum;

(f) 0.2% methyl paraben;

(g) 0.05% butyl paraben;

(h) 0.05% chlorhexidine gluconate;

(i) optionally 1.0% PVP; and

(j) water q.s to 100%.

A preferred composition comprises about:

(a) 50 mg/ml Clozapine;

(b) 150 mg/ml Sorbitol 70% crystallizing solution;

(c) 0.1 mg/ml Povidone K90;

(d) 7.8 mg/ml Sodium dihydrogen phosphate dihydrate;

(e) 0.48 to 0.66 mg/ml Sodium hydroxide;

(f) 2 mg/ml Sodium methylparaben;

(g) 0.2 mg/ml Sodium propylparaben;

(h) 182 mg/ml Glycerol;

(i) 2 mg/ml Xanthan gum; and

(j) 605.24 mg/ml Water.

A preferred composition comprises about:

(a) 50 mg/ml Clozapine;

(b) 150 mg/ml Sorbitol 70% crystallising solution;

(c) 10 mg/ml Povidone K90;

(d) 3.9 mg/ml Sodium dihydrogen phosphate dihydrate;

(e) 2 mg/ml Sodium methylparaben;

(f) 0.2 mg/ml Sodium propylparaben;

(g) 130 mg/ml Glycerol;

(h) 5.5 mg/ml Xanthan gum;

(i) 640.4 to 648.4 mg/ml Water;

(j) 0 to 4 mg/ml Sodium Hydroxide Solution (1M); and

(k) 0 to 4 mg/ml Hydrochloric acid Solution (1M).

In particular, the process to produce the composition may include the following steps:

(a) stirring the active ingredient clozapine with about three quarters of the propylene glycol ascribed to the batch;

(b) addition of the buffer salt (and optionally sweetening agents) dissolved in about half the volume of water ascribed to the batch with constant stirring;

(c) adjusting the pH value with the base component of the buffer with mixing;

(d) addition of the preservatives dissolved in the remaining propylene glycol;

(e) slow addition of the suspending agent with continuous stirring until the mixture thickens;

(f) optional addition of PVP dissolved in a portion of the remaining water ascribed to the batch with constant stirring; and

(g) further diluting the suspension with water to the desired end-volume.

The NaOH concentration used for adjustment of pH is preferably 4.6M.

Another preferred oral composition according to the invention includes:

(a) 50.0 mg/mL clozapine;

(b) 108.0 mg/mL glycerine;

(c) 4.2 mg/mL sodium dihydrogen phosphate dihydrate and sufficient sodium hydroxide to adjust the pH range from 6 to 9;

(d) 86.4 mg/mL sucrose;

(e) 5.2 mg/mL xanthan gum;

(f) 2.2 mg/mL methyl paraben;

(g) 0.2 mg/mL propyl paraben;

(h) 0.2 mg/mL butyl paraben;

(i) optionally 10.8 mg/mL PVP; and

(j) water q.s to 100% w/v (approx. 813 mg/mL).

The NaOH concentration used for adjustment of pH is preferably 0.1 M.

In particular, the process may comprise the following steps:

(a) stirring the active ingredient clozapine with about three quarters of the glycerine ascribed to the batch;

(b) addition of the buffer salt (and optionally sweetening agents) dissolved in about half the volume of water ascribed to the batch with constant stirring;

(c) adjusting the pH value with the base component of the buffer with mixing;

(d) addition of the preservatives dissolved in a small volume of water;

(e) slow addition of the suspending agent wetted with the remaining glycerine with continuous stirring until the mixture thickens;

(f) optional addition of PVP dissolved in a portion of the remaining water ascribed to the batch with constant stirring; and

(g) further diluting the suspension with water to the desired end-volume.

In particular, the process may comprise the following steps:

(a) wetting Clozapine with glycerine;

(b) adding PVP solution (0.025% w/v) and additional water;

(c) adding 0.25M buffer at pH 6.3 to the mixture;

(d) adding Semi hydrated xanthan gum/glycerol solution;

(e) adding Sorbitol and additional water;

(f) adding PVP solution (0.18% w/v) and additional water;

(g) adding Paraben/glycerine solution and additional water; and

(h) wherein the pH is between about 6.7 and 7.

In particular, the process may comprise the following steps:

(a) wetting Clozapine with glycerine;

(b) making a PVP solution at 0.025% w/v and adding to the mixture;

(c) adding additional water so that the concentration of PVP becomes 0.0125% w/v to promote flocculation;

(d) adding 0.25 M buffer at pH 6.3 to the mixture to enhance flocculation;

(e) adding Sorbitol 70% crystallizing solution and xanthan gum;

(f) adding additional PVP as a 0.18% solution;

(g) adding preservatives;

(h) The final concentration of PVP is 0.01% w/v and the buffer is 50 mM; and

(i) wherein the pH of the final suspension is about 6.9.

### EXAMPLES

The following examples are intended to illustrate the scope of the present invention in all its aspects but not to limit it thereto.

### Example 1

F1: Oral Suspension (pH=7.0)

| Ingredient | Quantity (mg) |
|---|---|
| Clozapine | 50 |
| Propylene Glycol (I) | 30 |
| Sodium Dihydrogen Phosphate Dihydrate | 7.8 |
| Sodium hydroxide | q.s ad pH = 7.0 |
| Sucrose | 80 |
| Xanthan Gum | 6.0 |
| Propylene Glycol (II) | 10 |
| Methyl Paraben | 2.0 |
| Butyl Paraben | 0.5 |
| Chlorhexidine Gluconate | 0.5 |
| Water | q.s ad 1 mL |

(1) Clozapine (50 mg) was mixed into a paste with Propylene Glycol (30 mg).

(2) Sodium Dihydrogen Phosphate Dihydrate (7.8 mg as a 1 M solution) was added to Fraction (1) with stirring.

(3) Sucrose (80 mg), dissolved in 0.35 mL of water was added to Fraction (2) with stirring.

(4) NaOH (4.6 mol/L) was added to Fraction (3) to adjust the pH to about 7.0.

(5) Methyl Paraben (2 mg) and Butyl Paraben (0.5 mg) were dissolved in Propylene Glycol (10 mg) with gentle warming.

(6) Fraction (5) was added slowly to Fraction (4) with constant stirring.

(7) Chlorhexidine Gluconate (0.5 mg) was dissolved in 0.2 mL of water.

(8) Fraction (7) was added to Fraction (6) with constant stirring.

(9) Xanthan Gum (6.0 mg) was slowly added to Fraction (8) with constant stirring taking care not to aerate the suspension.

(10) Fraction (9) was further diluted with water to 1 mL.

In a similar way there were prepared:

F2: Oral Suspension (pH=6.0±0.1)

| Ingredient | Quantity (mg) |
|---|---|
| Clozapine | 50 |
| Potassium Dihydrogen Phosphate Dihydrate | 27.2 |
| NaOH | q.s. ad pH = 6.0 |
| Sucrose | 80 |
| Xanthan Gum | 5.0 |
| Potassium Sorbate | 2.0 |
| Water | q.s. ad 1 mL |

F3: Oral Suspension (pH=7.0±0.1)

| Ingredient | Quantity (mg) |
|---|---|
| Clozapine | 50 |
| Potassium Dihydrogen Phosphate Dihydrate | 27.2 |
| NaOH | q.s. ad pH = 7.0 |
| Sucrose | 80 |
| Xanthan Gum | 5.0 |
| Potassium Sorbate | 2.0 |
| Water | q.s. ad 1 mL |

F4: Oral Suspension (pH=8.0±0.1)

| Ingredient | Quantity (mg) |
|---|---|
| Clozapine | 50 |
| Potassium Dihydrogen Phosphate Dihydrate | 27.2 |
| NaOH | q.s. ad pH = 8.0 |
| Sucrose | 80 |
| Xanthan Gum | 5.0 |
| Potassium Sorbate | 2.0 |
| Water | q.s. ad 1 mL |

### Example 2

The Table below summarizes the clozapine concentrations for formulations F1-F4 of Example 1 measured after a particular storage time of the composition at a particular temperature, expressed as the percentage of the initial clozapine concentration.

| Formulation | F1 | F2 | F3 | F4 |
|---|---|---|---|---|
| 1 month @ 5° C. | 102.6 | 102.6 | 101.0 | 100.2 |
| 1 month @ 50° C. | 98.6 | 100.6 | 101.1 | 102.7 |
| 3 month @ 40° C. | omitted | 104.7 | omitted | 97.8 |

### Example 3

25 mg/ml Clozapine Suspension pH 6.0

| Ingredients | mg |
|---|---|
| Sucrose | 80.0 |
| Methyl paraben | 0.5 |
| Propyl paraben | 0.1 |
| Clozapine | 25.0 |
| Hydroxypropylmethyl cellulose (HPMC) | 10.0 |
| Potassium dihydrogen phosphate | 24.0 |
| Water | 910.0 |

1. Dissolve the potassium dihydrogen phosphate in 910 mg of water and adjust the pH to 6.0.

2. Heat ⅓ of the phosphate buffer solution to 70° C., add methyl paraben and propyl paraben and dissolve.

3. Add with rapid stirring HPMC and mix for 2 minutes.

4. Add another ⅓ of the phosphate buffer solution and rapidly decrease the temperature of the mixture to room temperature continuing to stir for a further 15 minutes.

5. Add the remaining ingredients and continue to mix for a further 30 minutes.

Results

| | | | % of | |
|---|---|---|---|---|
| | | Assay | label | |
| Conditions | Description | (mg/ml) | claim | pH |
| Time Zero | A yellow suspension | 25.0 based on | 100.0 | 6.11 |
| | | compounding | | |
| 14 m/25° C./ | A yellow suspension and | 27.0 | 108.0 | 6.17 |
| 60% RH | settlement observed | | | |
| (RD4758) | but easily re-dispersed | | | |
| | after shaking | | | |
| 14 m/40° C. | A yellow suspension and | 25.4 | 101.6 | 6.02 |
| (RD4775) | settlement observed | | | |
| | but easily re-dispersed | | | |
| | after shaking | | | |

### Example 4

### 25 mq/ml Clozapine Suspension pH 8.0

| Ingredients | mg |
|---|---|
| Microcrystalline cellulose/sodium carboxymethyl cellulose | 12.0 |
| (Avicel RC 591) | |
| Hydroxypropylmethyl cellulose (HPMC) | 10.0 |
| Tween 80 | 5.0 |
| Clozapine | 25.0 |
| Methyl paraben | 0.5 |
| Propyl paraben | 0.1 |
| Sucrose | 80.0 |
| Potassium dihydrogen phosphate | 23.4 |
| Water | 884.0 |

1. Dissolve the potassium dihydrogen phosphate in 884 mg of water and adjust the pH to 8.0.

2. Heat ⅓ of the phosphate buffer solution to 70° C., add methyl paraben, propyl paraben and Tween 80 and dissolve.

3. Add with rapid stirring HPMC and mix for 2 minutes.

4. Add another ⅓ of the phosphate buffer solution and rapidly decrease the temperature of the mixture to room temperature continuing to stir for a further 15 minutes.

5. Slowly add AVICEL® RC 591 and continue stirring until visually smooth.

6. Add the remaining ingredients and continue to mix for a further 30 minutes.

Results

| | | Assay | | |
|---|---|---|---|---|
| Conditions | Description | (mg/ml) | % Yield | pH |
| Time Zero | A yellow suspension | 24.5 | 100.0 | 7.95 |
| 14 m/25° C./ | A yellow suspension and | 26.7 | 109.0 | 7.86 |
| 60% RH | settlement observed | | | |
| (RD4759) | but easily re-dispersed | | | |
| | after shaking | | | |
| 14 m/40° C. | A yellow suspension and | 28.2 | 115.1 | 7.46 |
| (RD4776) | settlement observed | | | |
| | but easily re-dispersed | | | |
| | after shaking | | | |

Results—Examples 3 and 4

The formulations of Examples 3 and 4 were stored at 40° C. and 25° C./60% RH for ˜14 months. The suspending agent in Example 3 was METOLOSE® (HPMC). The suspending agent in Example 4 was a combination of METOLOSE® (HPMC) and AVICEL® RC591. Both formulations did not show crystal growth at these pHs. Both formulations settle fairly quickly but were easily re-dispersed after shaking. The Clozapine assay results for both formulations show no degradation after 14 months and are thus defined as being physicochemically stable.

The formulation of Example 4 at pH 6 & 7 produced yellow crystals while stored at 25° C./60% RH and 40° C. for ˜14 months and are thus defined as being chemically unstable at this pH. Inclusion of PVP (crystal growth inhibitor) would be an option to correct this instability. However, the formulation of Example 4 at pH 8 and the formulation of Example 3 at pH 6 were shown to be physicochemically stable after ˜14 months at 40° C. and 25° C./60% RH.

### Example 5

### Alternatives to Sucrose (Xylitol, Sorbitol) in 50 mg/ml Clozapine Formulations

| | Formulation (mg/ml) | |
|---|---|---|
| Ingredients | Xylitol | Sorbitol |
| Xylitol | 86.4 | — |
| Sorbitol | — | 86.4 |
| PVP | 10.8 | 10.8 |
| Clozapine | 50.0 | 50.0 |
| Sodium Dihydrogen Phosphate dihydrate | 4.2 | 4.2 |
| Methyl Paraben (Na) | 2.2 | 2.2 |
| Propyl Paraben (Na) | 0.2 | 0.2 |
| Butyl Paraben (Na) | 0.2 | 0.2 |
| Xanthan gum | 5.2 | 5.2 |
| Glycerine | 140.4 | 140.4 |
| Water | 780.3 | 780.3 |
| Sodium Hydroxide | 0.1 | 0.1 |

1. Mix into a paste the Clozapine with % of the glycerine.

2. Dissolve Sodium Dihydrogen Phosphate Dihydrate in 3% of the required water and add to Fraction (1) with stirring.

3. Dissolve the sorbitol or xylitol and the three parabens in about 50% of the required water and add to Fraction (2) with stirring.

4. Add NaOH (1 mol/L) to Fraction (3) to adjust the pH to about 7.0. Add another 30% of the required water with stirring.

5. Wet the xanthan gum with ½ of the remaining glycerine. Add to Fraction (4) with stirring, rinsing the container with the remaining glycerine.

6. Dissolve the PVP with 5% of the required water and add to Fraction (5) with constant stirring.

7. Add the remaining water and continue to mix until the gum is well hydrated.

Results: 2 and 3 months stability at 40° C./75% RH (Results in ( ) are % of initial result).

| | | Initial | | 2 Months | | 3 Months | |
|---|---|---|---|---|---|---|---|
| | | | Assay % of | | Assay (% of | | Assay (% of |
| | Description | pH | label claim | pH | initial result) | pH | initial result) |
| Xylitol | Slight floculant settlement | 6.96 | 106.2% | 6.98 | (101.7%) | 6.76 | (105.3%) |
| | but readily re-disperses | | | | | | |
| | to a yellow suspension. | | | | | | |
| Sorbitol | Slight floculant settlement | 6.96 | 103.4% | 6.98 | (99.8%) | 6.80 | (100.0%) |
| | but readily re-disperses | | | | | | |
| | to a yellow suspension. | | | | | | |

Use of Xylitol or Sorbitol in place of sucrose would have advantages in the treatment of patients who are also diabetic. As shown in the above Example, formulations according to the invention that include xylitol or sorbitol in place of sucrose show acceptable stability characteristics.

### Example 6

### 50 mq/ml Clozapine Formulation. Trial Formulation 42

| | Amount |
|---|---|
| Ingredients | (mg/ml) |
| Sucrose | 86.4 |
| PVP | 10.8 |
| Clozapine | 50.0 |
| Sodium Dihydrogen Phosphate dihydrate | 4.2 |
| Methyl Paraben (Na salt) | 2.2 |
| Propyl Paraben (Na salt) | 0.2 |
| Butyl Paraben (Na salt) | 0.2 |
| Xanthan gum | 5.2 |
| Glycerine | 108.0 |
| Water | 811.6 |
| Banana Flavour canary | 1.1 |
| Sodium Hydroxide | 0.1 |

1. Mix into a paste the Clozapine with the glycerine.

2. Dissolve Sodium Dihydrogen Phosphate Dihydrate in 3% of the required water and add to Fraction (1) with stirring.

3. Dissolve the sucrose and the three parabens in about 50% of the required water and add to Fraction (2) with stirring.

4. Add NaOH (1 mol/L) to Fraction (3) to adjust the pH to about 7.0. Add another 30% of the required water with stirring.

5. With rapid stirring add the xanthan gum to Fraction (4).

6. Dissolve the PVP with 5% of the required water and add to Fraction (5) with constant stirring.

7. Add the remaining water and continue to mix until the gum is well hydrated.

Tables 1 and 2 below show the pharmacokinetic results obtained in a pilot scale bioequivalence Study of Trial Formulation 42 against a standard Clozapine tablet. It is considered that based on these data, when the usual number of subjects are used, the suspension will be shown to be bioequivalent to the tablet for the following reasons:

1. The absolute values of the mean AUC(0-inf) test and AUC(0-inf) reference are such that the ratio of AUC(0-inf) test/AUC(0-inf) is 91%. In the applicant's experience, if the ratio is >85% with six subjects then the study will pass when a statistically significant number of subjects is used.

2. The absolute values of the mean Cmax test and Cmax reference are such that the ratio of Cmax test/Cmax reference is 86%. In the applicant's experience if the ratio is >80% with six subjects then the study will pass when a statistically significant number of subjects is used.

3. It is thought that one cause of the mean ratios being <100% may be due to aeration of the suspension during preparation of the syringes for oral administration such that slightly less than 0.25 mL was administered. Processes for ensuring aeration of the suspension is kept to a minimum will be incorporated into syringe preparation in the larger studies.

4. The 90% CI for LogAUC(0-inf) has been determined to be 0.777-1.005. In the applicant's experience if the 90% CI is in the range 0.70-1.30 for six subjects then the study will pass when a statistically significant number of subjects is used.

5. The 90% CI for LogCmax has been determined to be 0.754-1.032. In the applicant's experience if the 90% CI is in the range 0.70-1.40 for six subjects then the study will pass when a statistically significant number of subjects is used. In fact the 90% CI for LogCmax at 0.754-1.032 is already in compliance with the wider interval of 0.75-1.33 allowed by the Note for Guidance on the Investigation of Bioequivalence and Bioavailability CPMP/EWP/QWP/1401/98. This Note is accepted as the guidance to be followed when conducting bioequivalence trials in either New Zealand or Australia.

TABLE 1
Summary of Pharmacokinetic Results Obtained - Formulation of Example 6

| | AUC_{0-∞} | AUC₀₋ₜ | Cmax | Tmax | t½ |
|---|---|---|---|---|---|
| | (ng · hr/ml) | (ng · hr/ml) | (ng/ml) | (hr) | (hr) |
| | Mean | Mean | Mean | Mean | Mean |
| | S.D | S.D | S.D | S.D | S.D |
| | Range | Range | Range | Range | Range |
| Clozapine 50 mg/ml | 260.75 | 250.31 | 28.78 | 1.42 | 15.80 |
| suspension (T) | 117.08 | 114.48 | 10.40 | 0.20 | 4.27 |
| 0.25 mL | (112.50-448.72) | (105.88-438.95) | (16.80-46.90) | (1.00-1.52) | (8.85-20.65) |
| B: Trial 42 | | | | | |
| (Douglas, | | | | | |
| New Zealand) | | | | | |
| Clozapine 25 mg | 286.64 | 275.52 | 33.62 | 1.83 | 15.15 |
| tablets (R) ½ tablet | 99.49 | 94.33 | 14.13 | 1.58 | 4.15 |
| B: 001G8380T | (142.64-390.13) | (139.96-373.34) | (15.40-55.90) | (0.98-5.02) | (8.67-20.77) |
| (Novartis, USA) | | | | | |
| Mean Ratio¹ | 90.97 | 90.85 | 85.62 | 77.27 | 104.30 |
| Geometric Ratio¹ | 88.39 | 87.85 | 88.21 | 93.87 | — |

¹Mean Ratio = Mean (T)/Mean (R)

TABLE 2
Clozapine Bioequivalence Summary Statistics for
the Example 6 Formulation and Clozapine Tablets

| Variable | Anova | 90% C.I. |
|---|---|---|
| Log₁₀ (AUC₀-∞) | 0.111 | (0.777, 1.005)* |
| Log₁₀ (AUC0-t) | 0.094 | (0.774, 0.997) |
| Log₁₀ (Cmax) | 0.164 | (0.754, 1.032)* |
| AUC₀-∞ | 0.254 | (0.765, 1.054) |
| AUC0-t | 0.242 | (0.766, 1.051) |
| Cmax | 0.103 | (0.711, 1.002) |
| Tmax | 0.518 | (0.089, 1.457) |
| Tmax⁺ | Significant difference | (0.547, 1.513) |
| t½ | 0.423 | (0.909, 1.177) |

*Criteria used to assess Bioequivalence, i.e. 90% CI between 0.80 and 1.25 for AUC_{0-∞} and Cmax
⁺Nonparametic Analysis

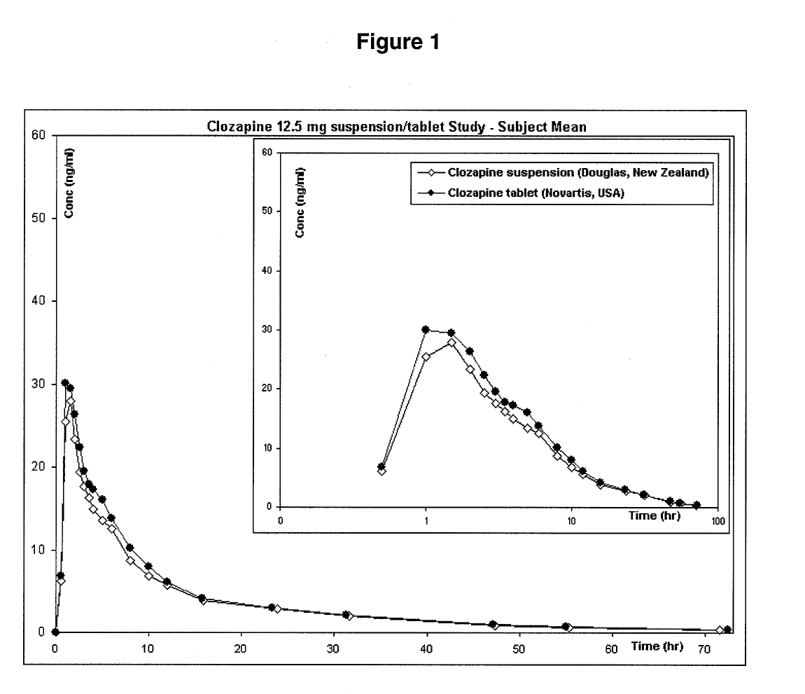

Tabulated data showing the mean plasma concentration versus time of Trial Formulation 42 (Example 6) and a Clozapine tablet are detailed in FIG. 1.

### Example 7

### Clozapine Suspension Stability over pH Range

Method of Preparation of Clozapine Formulations used in Table 3 below:

1. Clozapine (20 g) was combined with 50 g of a phosphate buffer I (pH 3) or phosphate buffer II (pH 5, 6, 7, 11).

2. A further 350 g of phosphate buffer I (pH 3) or phosphate buffer II (pH=5, 6, 7, 11) was then added to the mixture from 1.

3. The pH of the mixture from 2 was adjusted to the desired value by the addition of concentrated phosphoric acid or sodium hydroxide as appropriate.

4. Additional buffer was added to the mixture from 3 to a final mass of 400 g (5% w/w clozapine).

Preparation of the buffer solutions was as follows:

Phosphate buffer I

3.4 g of potassium dihydrogen phosphate was dissolved in 900 mL of water. The pH was adjusted to 3.0 with phosphoric acid and the resulting solution diluted to 1000 mL.

Phosphate buffer II

18.72 g of sodium dihydrogen phosphate was dissolved in water and made to a final volume of 2 L.

Table 3 below shows stability data for aqueous suspensions of clozapine at a range of pH values.

TABLE 3

| | pH 3 | | pH 5 | | pH 6 | |
|---|---|---|---|---|---|---|
| | Initial | 5 days/70° C. | Initial | 5 days/70° C. | Initial | 5 days/70° C. |
| | Testing | Storage | Testing | Storage | Testing | Storage |
| Description | Yellow powder | Orange powder | Yellow powder | Yellow powder | Yellow powder | Yellow powder |
| | in yellow | in red | in yellow | in dark yellow | in yellow | in yellow |
| | solution | solution | solution | solution | solution | solution |
| pH | 3.01 | 3.86 | 5.20 | 5.70 | 5.99 | 6.20 |
| Total Unknown | 0.19 | 4.14 | 0.00 | 0.07 | 0.00 | 0.04 |
| Impurities % | | | | | | |
| Impurity A | 0.10 | 36.70 | 0.00 | 0.80 | 0.00 | 0.52 |
| (CDD)% | | | | | | |
| Total | 0.29 | 40.84 | 0.00 | 0.87 | 0.00 | 0.56 |
| impurities % | | | | | | |

| | |
|---|---|
| pH 7 | pH 11 |

| | Initial | 5 days/70° C. | Initial | 5 days/70° C. |
|---|---|---|---|---|
| | Testing | Storage | Testing | Storage |
| Description | Yellow powder | Yellow powder | Yellow powder | Yellow powder |
| | in yellow | in yellow | in light yellow | in light yellow |
| | solution | solution | coloured solution | coloured solution |
| pH | 6.95 | 6.54 | 11.08 | 10.54 |
| Total Unknown | 0.00 | 0.00 | 0.00 | 0.00 |
| Impurities % | | | | |
| (Impurity A | 0.00 | 0.10 | 0.00 | 0.00 |
| (CDD)% | | | | |
| Total | 0.00 | 0.10 | 0.00 | 0.00 |
| impurities % | | | | |

Results at 70° C. after 5 days indicate that the clozapine molecule is very stable with respect to related substances with no degradation observed when the pH is controlled between pH 6 and pH 11. At pH 3 clozapine degrades rapidly, with degradants present at a level of about 41%, after 5 days at 70° C. At pH 5 the level of growth impurity A (CDD “8-chloro-5H-Dibenzo-[b,e]-1,4-Diazepine-11-one) at 0.8% is unacceptably high as the limit for impurities according to the ICH guidelines for this product would be 0.2%.

### Example 8

### Clozapine Oral Suspension

| Ingredients | mg/ml | % w/v |
|---|---|---|
| Clozapine | 50 | 5 |
| Sorbitol 70% | 150 | 15 |
| crystallising | | |
| solution | | |
| Povidone K90 | 10 | 1 |
| Sodium | 3.9 | 0.39 |
| dihydrogen | | |
| phosphate | | |
| dihydrate | | |
| Sodium | 2 | 0.2 |
| methylparaben | | |
| Sodium | 0.2 | 0.02 |
| propylparaben | | |
| Glycerol | 130 | 13 |
| Xanthan gum | 5.5 | 0.55 |
| Water | 640.4 to 648.4 | 64.04 to 64.84 |
| Sodium | 0 to 4 | 0 to 0.4 |
| Hydroxide | | |
| Solution (1M) | | |
| Hydrochloric | 0 to 4 | 0 to 0.4 |
| acid Solution | | |
| (1M) | | |

Manufacturing Procedure for Example 8

Manufacturing procedure for Clozapine 50 mg/ml Suspension

| STEP | INSTRUCTION |
|---|---|
| 1 | Dissolve PVP with 13.7% of the required water. |
| 2 | Dissolve sodium dihydrogen phosphate dihydrate with 3.4% of the |
| | required water. |
| 3 | Mix Clozapine with 76.9% of the required glycerine and mix until |
| | Clozapine is fully wetted. |
| 4 | Add the buffer solution from Step 2 to the Clozapine mix in Step |
| | 3 and mix well. |
| 5 | In the main vessel, dissolve the parabens with 16.5% of the |
| | required water. Mix well |
| 6 | To the main vessel in Step 5 add the Clozapine/glycerine buffer |
| | mix from Step 4 and mix well |
| 7 | Add 11% of the required water and mix well. |
| 8 | Add to the Clozapine mix in Step 7 add sorbitol crystallising |
| | solution and rinse with 6.7% of the required water and mix. |
| 9 | Mix xanthan gum with 15.4% of the required glycerine and mix. |
| | While mixing add 27.5% of the required water and the remaining |
| | glycerol and continue to mix until the xanthan gum is hydrated. |
| 10 | Add the xanthan gum from Step 9 into the Clozapine mix from |
| | Step 8 adding 13.7% of the required water and mix well using |
| | a high speed mixer. |
| 11 | Add the PVP solution from Step 1 to the Clozapine mix in Step |
| | 10 adding 6.2% of the required water and mix well. |
| 12 | Check the pH of the suspension is between pH 6.6 and 6.8 |
| 13 | Add the remaining water and mix well. |

### Example 9

### Clozapine Oral Suspension

| Ingredients | mg/ml | % w/v |
|---|---|---|
| Clozapine | 50 | 5 |
| Sorbitol 70% | 150 | 15 |
| crystallising | | |
| solution | | |
| Povidone K90 | 0.01 | 0.001 |
| (1) | | |
| Povidone K90 | 0.09 | 0.009 |
| (2) | | |
| Sodium | 7.8 | 0.78 |
| dihydrogen | | |
| phosphate | | |
| dihydrate | | |
| Sodium | 0.48 to 0.66 | 0.048 to 0.066 |
| hydroxide | | |
| Sodium | 2 | 0.2 |
| methylparaben | | |
| Sodium | 0.2 | 0.02 |
| propylparaben | | |
| Glycerol | 182 | 18.2 |
| Xanthan gum | 2 | 0.2 |
| Water | 705.24 | 70.524 |

Manufacturing Procedure for Example 9

Manufacturing procedure for Clozapine 50 mg/ml Suspension:

| STEP | INSTRUCTION |
|---|---|
| 1 | Dissolve PVP (1) with 5.7% of the required water. |
| 2 | Dissolve sodium dihydrogen phosphate dihydrate and sodium |
| | hydroxide with 28.4% of the required water. pH of the buffer |
| | to be within pH 6.2 and 6.35. |
| 3 | Mix Clozapine with 41.7% of the required glycerine and mix |
| | until Clozapine is fully wetted. |
| 4 | Add PVP (1) solution from Step 1 to the Clozapine mix in Step |
| | 3 adding additional 5.7% of the required water and mix. |
| 5 | Add the buffer solution from Step 2 to the Clozapine mix in Step |
| | 4 and mix well. |
| 6 | Add to the Clozapine mix in Step 5 add sorbitol crystallising |
| | solution and 4.3% of the required water and mix. |
| 7 | Mix xanthan gum with 16.5% of the required glycerine and mix. |
| | While mixing add 28.4% of the required water and continue to |
| | mix until the xanthan gum is hydrated. |
| 8 | Add the xanthan gum from Step 7 into the Clozapine mix from |
| | Step 6 adding 8.2% of the required water and mix well using |
| | a high speed mixer. |
| 9 | Dissolve PVP (2) with 7.1% of the required water. |
| 10 | Add the PVP (2) solution from Step 9 to the Clozapine mix in |
| | Step 8 adding 4.3% of the required water and mix well. |
| 11 | Dissolve the parabens with 2.8% of the required water and add |
| | the remaining glycerine. Mix well. |
| 12 | Add the paraben mix from Step 11 to the Clozapine mix in Step |
| | 10 adding the remaining required water. Mix using the high |
| | speed mixer for 30 minutes. |
| 13 | Check the pH of the suspension is between pH 6.7 and 7.0 |

Administration of the Suspension

It is envisaged that the product would be supplied in a glass or plastic container with a child proof closure together with a syringe marked in mL for ease of dosing. The minimum marked volume of the syringe would be 0.25 mL to allow for accurate dosing of the recommended starting dose of 12.5 mg based on the Clozapine 50 mg/mL product. The maximum volume of the syringe would be around 10 mL to allow ready dispensing of the range of most therapeutic doses in one application. The syringe should be emptied into a non-alcoholic drink with stirring. Orange juice, coffee and some carbonated soft drinks are suitable. The syringe should be rinsed and dried after use.

### Example 10

### Evaluation of Clozapine Stability from pH 6 to pH 11

Clozapine suspension formulations were made and tested for stability. Table 4 sets forth the description of the suspensions made between pH 7 and 11 with phosphate buffers. Table 5 sets forth the description of the suspension at pH 6 with citric acid, and the description of the suspensions made between pH 9 and 11 with carbonate buffers. These Tables show that the suspensions remain acceptably stable when stored at either 25° C./60% RH or 40° C./75% RH. By acceptably stable is meant that the pH and clozapine assay remained within specified ranges and the clozapine remained in suspension or was able to be readily resuspended by shaking. A slight change in the description is noted for the suspensions made with either phosphate buffer or citric acid buffer at pH 6 after 3 months storage at 40° C./75% RH (see Table 4 and Table 5). At this condition the supernatant of the suspension was brownish in colour and the suspension took a longer time to resuspend when compared to the other suspensions.

pH: The pH of the suspensions from pH 8 to 11 all showed a drop in pH when stored for 3 months at either 25° C./60% RH or 40° C./75% RH. However the pH remained within the specified range.

Clozapine Assay: The assay of clozapine remained within the specifications for all suspensions prepared. A small drop in the assay was observed for those suspensions prepared at pH 6 and pH 9 after storage for 3 months at 40° C./75% RH.

TABLE 4

| | | Buffer Composition | | | | | |
|---|---|---|---|---|---|---|---|
| | | Sodium dihydrogen phosphate dihydrate/NaOH buffer | | | | | |
| | | Batch No. | | | | | |
| | | F85 | F86 | F87 | F88 | F89 | P90 |
| Tests | Time | pH 6 | pH 7 | pH 8 | pH 9 | pH 10 | pH 11 |
| Description | 0 | A yellow | A yellow | A yellow | A yellow | A yellow | A yellow |
| Specification: | | free flowing | free flowing | free flowing | free flowing | free flowing | free flowing |
| A yellow free | | suspension, | suspension, | suspension, | suspension, | suspension, | suspension, |
| flowing | | free from | free from | free from | free from | free from | free from |
| suspension, | | particulate | particulate | particulate | particulate | particulate | particulate |
| free from | | matter, free | matter, free | matter, free | matter, free | matter, free | matter, free |
| particulate | | from foreign | from foreign | from foreign | from foreign | from foreign | from foreign |
| matter, free | | matter | matter | matter | matter | matter | matter |
| from foreign | 3M | A yellow | A yellow | A yellow | A yellow | A yellow | A yellow |
| matter | 25° C./ | free flowing | free flowing | free flowing | free flowing | free flowing | free flowing |
| | 60% RH | suspension, | suspension, | suspension, | suspension, | suspension, | suspension, |
| | | free from | free from | free from | free from | free from | free from |
| | | particulate | particulate | particulate | particulate | particulate | particulate |
| | | matter, free | matter, free | matter, free | matter, free | matter, free | matter, free |
| | | from foreign | from foreign | from foreign | from foreign | from foreign | from foreign |
| | | matter. 10 sec | matter. 10 sec | matter. 10 sec | matter. 10 sec | matter. 10 sec | matter. 10 sec |
| | | to fully | to fully | to fully | to fully | to fully | to fully |
| | | suspend with | suspend with | suspend with | suspend with | suspend with | suspend with |
| | | vigorous shaking, | vigorous shaking, | vigorous shaking, | vigorous shaking, | vigorous shaking, | vigorous shaking, |
| | | less aeration | less aeration | less aeration | less aeration | less aeration | less aeration |
| | 3M | A brownish free | A yellow | A yellow | A yellow | A yellow | A yellow |
| | 40° C./ | flowing suspension, | free flowing | free flowing | free flowing | free flowing | free flowing |
| | 75% RH | free from particulate | suspension, | suspension, | suspension, | suspension, | suspension, |
| | | matter, free from | free from | free from | free from | free from | free from |
| | | foreign matter. 40 | particulate | particulate | particulate | particulate | particulate |
| | | to 50 sec to fully | matter, free | matter, free | matter, free | matter, free | matter, free |
| | | resuspend with | from foreign | from foreign | from foreign | from foreign | from foreign |
| | | vigorous shaking, | matter. 10 sec | matter. 10 sec | matter. 10 sec | matter. 10 sec | matter. 10 sec |
| | | high aeration, | to fully | to fully | to fully | to fully | to fully |
| | | brownish clear | suspend with | suspend with | suspend with | suspend with | suspend with |
| | | supernatant | vigorous shaking, | vigorous shaking, | vigorous shaking, | vigorous shaking, | vigorous shaking, |
| | | before shaking | less aeration | less aeration | less aeration | less aeration | less aeration |
| | 0 | 6.06 | 7.03 | 8.05 | 8.88 | 9.59 | 10.83 |
| pH | 3M | 6.09 | 6.98 | 7.80 | 8.39 | 8.79 | 10.27 |
| Specification: | 25° C./ | | | | | | |
| Record only | 60% RH | | | | | | |
| | 3M | 6.02 | 6.86 | 7.46 | 7.77 | 8.03 | 9.91 |
| | 40° C./ | | | | | | |
| | 75% RH | | | | | | |
| Assay | 0 | 50.3 mg/ml | 50.4 mg/ml | 49.6 mg/ml | 49.6 mg/ml | 49.4 mg/ml | 48.8 mg/ml |
| Clozapine | | 100.6% | 100.8% | 99.2% | 99.2% | 98.8% | 97.6% |
| Specification: | 3M | 50.0 mg/ml | 50.5 mg/ml | 49.6 mg/ml | 49.9 mg/ml | 49.7 mg/ml | 48.7 mg/ml |
| 47.5 to 52.5 | 25° C./ | 100.0% | 101.0% | 99.2% | 99.8% | 99.4% | 97.4% |
| mg/ml | 60% RH | | | | | | |
| 95.0 to | 3M | 49.3 mg/ml | 50.5 mg/ml | 49.9 mg/ml | 48.8 mg/ml | 49.7 mg/ml | 48.8 mg/ml |
| 105.0% of | 40° C./ | 98.6% | 101.0% | 99.8% | 97.6% | 99.4% | 97.6% |
| label claim | 75% RH | | | | | | |

TABLE 5

| | | Buffer Composition | | |
|---|---|---|---|---|
| | | Citric acid/Sodium | Sodium carbonate/Sodium | |
| | | citrate buffer | bicarbonate buffer | |
| | | Batch No. | | |
| | | F91 | F92 | F93 |
| Tests | Time | pH 6 | pH 9 | pH 11 |
| Description | 0 | A yellow free | A yellow free | A yellow free |
| Specification: | | flowing suspension, | flowing suspension, | flowing suspension, |
| A yellow free | | free from particulate | free from particulate | free from particulate |
| flowing suspension, | | matter, free from | matter, free from | matter, free from |
| free from particulate | | foreign matter | foreign matter | foreign matter |
| matter, free from | 3M | A yellow free | A yellow free | A yellow free |
| foreign matter | 25° C./60% RH | flowing suspension, | flowing suspension, | flowing suspension, |
| | | free from particulate | free from particulate | free from particulate |
| | | matter, free from | matter, free from | matter, free from |
| | | foreign matter. | foreign matter. | foreign matter. |
| | | 10 sec to fully suspend | 10 sec to fully suspend | 10 sec to fully suspend |
| | | with vigorous shaking, | with vigorous shaking, | with vigorous shaking, |
| | | less aeration | less aeration | less aeration |
| | 3M | A brownish free | A yellow free | A yellow free |
| | 40° C./75% RH | flowing suspension, | flowing suspension, | flowing suspension, |
| | | free from particulate | free from particulate | free from particulate |
| | | matter, free from | matter, free from | matter, free from |
| | | foreign matter. | foreign matter. | foreign matter. |
| | | 40 to 50 sec to fully | 10 sec to fully suspend | 10 sec to fully suspend |
| | | resuspend with vigorous | with vigorous shaking, | with vigorous shaking, |
| | | shaking, high aeration, | less aeration | less aeration |
| | | brownish clear supernatant | | |
| | | before shaking | | |
| pH | 0 | 6.18 | 8.95 | 10.92 |
| Specification: | 3M | 6.21 | 8.48 | 10.14 |
| Record only | 25° C./60% RH | | | |
| | 3M | 6.21 | 7.80 | 9.90 |
| | 40° C./75% RH | | | |
| Assay | 0 | 49.9 mg/ml | 49.5 mg/ml | 49.6 mg/ml |
| Clozapine | | 99.8% | 99.0% | 99.2% |
| Specification: | 3M | 50.1 mg/ml | 49.5 mg/ml | 49.1 mg/ml |
| 47.5 to 52.5 mg/ml | 25° C./60% RH | 100.2% | 99.0% | 98.2% |
| 95.0 to 105.0% | 3M | 49.2 mg/ml | 49.8 mg/ml | 49.6 mg/ml |
| | 40° C./75% RH | 98.4% | 99.6% | 99.2% |

### Example 11

### Sugar Suspension A

A high sugar composition may be prepared by known mixing techniques with the following ingredients:

| | |
|---|---|
| Sorbitol 70% solution | 94.3% w/w (equivalent to 66.0% sorbitol) |
| PVP K90 | 0.01% w/w |
| Clozapine | 5.0% w/w |
| Water | QS to 100% |

### Example 12

### Sugar Suspension B

A high sugar composition may be prepared by known mixing techniques with the following ingredients:

| | |
|---|---|
| Sucrose | 66.0% w/w |
| PVP K90 | 0.01% w/w |
| Clozapine | 5.0% w/w |
| Water | QS to 100% |

Suspensions prepared according to Examples 11 and 12 were stored for 3 months at 40° C./RH75%. They were assessed against the following acceptance criteria:
Description: A free flowing yellow suspension free from particulate matter and foreign matter
Clozapine assay: 95.0% to 105.0% w/w
pH: 5.6 to 11

The results are set out in Table 6.

TABLE 6

| | | | Clozapine | |
|---|---|---|---|---|
| | | | assay (% | |
| Formulation | Storage | Description | w/w) | pH |
| Sorbitol | Initial | A free flowing yellow | 99.4% | 7.11 |
| (Example 11) | | suspension free from | | |
| | | particulate matter and | | |
| | | foreign matter | | |
| | 3M | A free flowing yellow | 88.2% | 6.40 |
| | 40/75 | suspension free from | | |
| | | particulate matter and | | |
| | | foreign matter | | |
| Sucrose | Initial | A crust on top of the | 101.8% | 7.70 |
| (Example 12) | | formulation was observed. | | |
| | | Afte [symbol] breaking the crust | | |
| | | the suspension was free | | |
| | | flowing, bu [symbol] thick. | | |
| | | Yellow in colour | | |
| | 3M | A free flowing yellow | 103.6% | 7.40 |
| | 40/75 | suspension free from | | |
| | | particulate matter and | | |
| | | foreign matter | | |

[symbol]

indicates data missing or illegible when filed

Related substances levels were acceptable after storage.

The foregoing describes the invention including preferred forms thereof, alterations or modifications as would be obvious to a person skilled in this particular art are intended to be included within the scope of the invention as defined in the attached claims.

1. A physicochemically stable aqueous composition for oral administration comprising clozapine in suspension and an agent capable of controlling and/or maintaining the pH of the composition, wherein the pH of the composition is maintained within the range of about 5.6 to about 11.

2. The composition according to claim 1 wherein the agent is a buffer system.

3. A physicochemically stable aqueous composition according to claim 1 comprising clozapine in suspension, a wetting agent, a suspending agent, and a buffer.

4. The composition according to claim 1 wherein the buffer is a sodium phosphate/sodium hydroxide buffer.

5. The composition according to claim 1 wherein the pH is maintained in the range of from about 5.9 to about 7.

6. The composition according to claim 1 wherein the pH is maintained in the range of from about 5.6 to about 8.

7. The composition according to claim 1 wherein the amount of clozapine in the composition is from about 0.1% to about 10% w/v of the total composition.

8. The composition according to claim 3 wherein the wetting agent is present in an amount of between about 0.1% and about 19% w/v.

9. The composition according to claim 3 wherein the wetting agent is selected from any one or more of propylene glycol, glycerin, or polyethylene glycol.

10. The composition according to claim 3 wherein the suspending agent is selected from any one or more of xanthan gum, guar gum, tragacanth gum, hydroxypropylmethylcellulose or microcrystalline cellulose.

11. The composition according to claim 10 wherein the suspending agent is present in an amount of between about 0.1% and about 2.0% w/w.

12. The composition according to claim 10 wherein the suspending agent is xanthan gum.

13. The composition according to claim 1 or claim 3 further comprising polyvinyl pyrrolidone (PVP).

14. The composition according to claim 13 wherein the PVP is a long-chain PVP and is present in an amount of between about 0.005% and 2.0% by weight based on the total volume of the composition.

15. The composition according to claim 1 or claim 3 further comprising a preservative selected from any one or more of methyl, propyl, butyl parabens, and combinations thereof.

16. The composition according to claim 15 wherein the preservative is a mixture of methyl and propyl parabens.

17. The composition according to claim 13 wherein each preservative is present in an amount of between about 0.01% and about 0.5% w/v.

18. The composition according to claim 3 wherein the composition further includes a sweetening agent and/or a flavouring substance.

19. The composition according to claim 1 or claim 3 wherein the composition comprises: clozapine, glycerine, sodium dihydrogen phosphate dihydrate/NaOH buffer, xanthan gum, methyl paraben, propyl paraben, PVP90 and water.

20. The composition according to claim 1 comprising clozapine in suspension and a sweetening agent.

21. The composition according to claim 20 wherein the sweetening agent is selected from sucrose or sorbitol.

22. A method for preparing a physicochemically stable aqueous composition including clozapine in suspension, the method comprising the step of controlling the pH of the formulation between about 5.6 and about 11 using a buffer.

23. The method according to claim 22 wherein the pH is between 5.6 and 8.

24. The method according to claim 22 wherein the pH is between 5.9 and 7.

25. The method according to claim 22 wherein the buffer concentration before addition to the clozapine is between about 0.1M and about 0.5M.

26. The method according to claim 22 wherein the method further includes the addition of a long chain PVP.

27. A method for preparing a physicochemically stable aqueous composition comprising clozapine in suspension, a wetting agent comprising propylene glycol, a buffer, one or more preservatives and a suspending agent, comprising the following steps:
(a) stirring the clozapine with about three quarters of the propylene glycol ascribed to the batch;
(b) addition of the buffer salt (and optionally sweetening agents) dissolved in about half the volume of water ascribed to the batch with constant stirring;
(c) adjusting the pH value of the batch with the base component of the buffer with mixing;
(d) addition to the batch of the preservatives dissolved in the remaining propylene glycol;
(e) slow addition of the suspending agent to the batch with continuous stirring until the mixture thickens; and,
(f) further diluting the suspension with water to the desired end-volume.

28. A method for producing a physicochemically stable aqueous composition comprising clozapine in suspension, a wetting agent comprising glycerine, a buffer, one or more preservatives and a suspending agent, comprising the following steps:
(a) stirring the clozapine with about three quarters of the glycerine ascribed to the batch;
(b) addition of the buffer salt (and optionally sweetening agents) dissolved in about half the volume of water ascribed to the batch with constant stirring;
(c) adjusting the pH value with the base component of the buffer with mixing;
(d) addition of the preservatives dissolved in a small volume of water;
(e) slow addition of the suspending agent wetted with the remaining glycerine with continuous stirring until the mixture thickens; and
(f) further diluting the suspension with water to a desired end-volume.

29. The method according to claim 27 or 28 wherein PVP is added as an aqueous solution following addition of the suspending agent.

30. A method for preparing a physicochemically stable aqueous composition comprising clozapine in suspension, a wetting agent comprising glycerine, a buffer, a suspending agent comprising xanthan gum, sorbitol, and PVP, comprising the following steps:
(a) wetting Clozapine with glycerine;
(b) adding PVP solution (0.025% w/v) and additional water;
(c)adding 0.25 M buffer at pH 6.3 to the mixture;
(d) adding Semi hydrated xanthan gum/glycerol solution;
(e) adding Sorbitol and additional water;
(f) adding PVP solution (0.18% w/v) and additional water;
(g) adding Paraben/glycerine solution and additional water;
(h) wherein the pH of the final suspension is between about 6.7 and 7.

31. A method for producing a physicochemically stable aqueous composition including clozapine in suspension, a wetting agent comprising glycerine, a buffer, a suspending agent comprising xanthan gum, sorbitol, PVP, and one or more preservatives, comprising the following steps:
(a) wetting Clozapine with glycerine;
(b) making a PVP solution at 0.025% w/v and adding to the mixture;
(c) adding additional water so that the concentration of PVP becomes 0.0125% w/v to promote flocculation;
(d) adding 0.25 M buffer at pH 6.3 to the mixture to enhance flocculation;
(e) adding Sorbitol 70% crystallizing solution and xanthan gum;
(f) adding additional PVP as a 0.18% w/v solution;
(g) adding preservatives;
wherein the pH of the final suspension is about 6.9, and the final concentration of PVP is 0.01% and the buffer is 50 mM.

32. A physicochemically stable aqueous composition comprising about:
(a) 50 mg/ml Clozapine in suspension
(b) 150 mg/ml Sorbitol 70% crystallizing solution
(c) 0.1 mg/ml Povidone K90
(d) 7.8 mg/ml Sodium dihydrogen phosphate dihydrate
(e) 0.48 to 0.66 mg/ml Sodium hydroxide
(f) 2 mg/ml Sodium methylparaben
(g) 0.2 mg/ml Sodium propylparaben
(h) 182 mg/ml Glycerol
(i) 2 mg/ml Xanthan gum
(j) q.s. Water.

33. A physicochemically stable aqueous composition comprising about:
(a) 50 mg/ml Clozapine in suspension
(b) 150 mg/ml Sorbitol 70% crystallizing solution
(c) 10 mg/ml Povidone K90
(d) 3.9 mg/ml Sodium dihydrogen phosphate dihydrate
(e) 2 mg/ml Sodium methylparaben
(f) 0.2 mg/ml Sodium propylparaben
(g) 130 mg/ml Glycerol
(h) 5.5 mg/ml Xanthan gum
(i) 0 to 4 mg/ml Sodium Hydroxide Solution (1M)
(j) 0 to 4 mg/ml Hydrochloric acid Solution (1M)
(k) q.s. Water.

34. A physicochemically stable aqueous composition for oral administration comprising clozapine in suspension, a wetting agent, a stabilizing agent, and a buffer, wherein the pH of the composition is maintained within the range of about 6 to about 11.

35. The composition according to claim 34, wherein the wetting agent is any one or more of propylene glycol, glycerin, or polyethylene glycol.

36. The composition according to claim 34, wherein the stabilizing agent is any one or more of xanthan gum, guar gum, tragacanth gum, hydroxypropyl methylcellulose, or microcrystalline cellulose.

37. A physicochemically stable aqueous composition consisting of about:
(a) 50 mg/ml Clozapine in suspension
(b) 150 mg/ml Sorbitol 70% crystallizing solution
(c) 0.1 mg/ml Povidone K90
(d) 7.8 mg/ml Sodium dihydrogen phosphate dihydrate
(e) 0.48 to 0.66 mg/ml Sodium hydroxide
(f) 2 mg/ml Sodium methylparaben
(g) 0.2 mg/ml Sodium propylparaben
(h) 182 mg/ml Glycerol
(i) 2 mg/ml Xanthan gum
(j) q.s. Water .

38. A physicochemically stable aqueous composition consisting of about:
(a) 50 mg/ml Clozapine in suspension
(b) 150 mg/ml Sorbitol 70% crystallizing solution
(c) 10 mg/ml Povidone K90
(d) 3.9 mg/ml Sodium dihydrogen phosphate dihydrate
(e) 2 mg/ml Sodium methylparaben
(f) 0.2 mg/ml Sodium propylparaben
(g) 130 mg/ml Glycerol
(h) 5.5 mg/ml Xanthan gum
(i) 0 to 4 mg/ml Sodium Hydroxide Solution (1M)
(j) 0 to 4 mg/ml Hydrochloric acid Solution (1M)
(k) q.s. Water.

39. A physicochemically stable aqueous composition consisting essentially of about:
(a) 50 mg/ml Clozapine in suspension
(b) 150 mg/ml Sorbitol 70% crystallizing solution
(c) 0.1 mg/ml Povidone K90
(d) 7.8 mg/ml Sodium dihydrogen phosphate dihydrate
(e) 0.48 to 0.66 mg/ml Sodium hydroxide
(f) 2 mg/ml Sodium methylparaben
(g) 0.2 mg/ml Sodium propylparaben
(h) 182 mg/ml Glycerol
(i) 2 mg/ml Xanthan gum
(j) q.s. Water.

40. A physicochemically stable aqueous composition consisting essentially of about:
(a) 50 mg/ml Clozapine in suspension
(b) 150 mg/ml Sorbitol 70% crystallizing solution
(c) 10 mg/ml Povidone K90
(d) 3.9 mg/ml Sodium dihydrogen phosphate dihydrate
(e) 2 mg/ml Sodium methylparaben
(f) 0.2 mg/ml Sodium propylparaben
(g) 130 mg/ml Glycerol
(h) 5.5 mg/ml Xanthan gum
(i) 0 to 4 mg/ml Sodium Hydroxide Solution (1M)
(j) 0 to 4 mg/ml Hydrochloric acid Solution (1M)
(k) q.s. Water.

41. A method of treating a patient in need of such treatment with an antipsychotic comprising administering the composition of claim 1 to the patient.

42. A method of treating a patient in need of such treatment with an antipsychotic comprising:
adding the physicochemically stable aqueous composition of clozapine of claim 1 to a non-alcoholic drink with stirring; and
orally administering the drink to the patient.

43. A method of administration of a physicochemically stable aqueous composition of clozapine comprising orally administering the composition of claim 1 to a patient in need of such treatment.

44. A method of administration of the physicochemically stable aqueous composition of clozapine of claim 1 comprising:
adding the clozapine composition to a non-alcoholic drink with stirring; and
administering the drink to a patient in need of such treatment.
